(19) United States
(12) Patent Application Publication
(10) Pub. No.: US 2016/0243021 A1
(43) Pub. Date: Aug. 25, 2016

(54) Collagen-Boosting Compositions And Methods

(71) Applicant: ELC MANAGEMENT LLC, MELVILLE, NY (US)

(72) Inventors: DONALD F. COLLINS, PLAINVIEW, NY (US); NADINE A. PERNODET, HUNTINGTON STATION, NY (US); KENNETH D. MARENUS, DIX HILLS, NY (US)

(21) Appl. No.: 15/019,286

(22) Filed: Feb. 9, 2016

Related U.S. Application Data
(60) Provisional application No. 62/118,038, filed on Feb. 19, 2015.

(51) Int. Cl. A61K 8/97; A61K 8/55; A61Q 19/08; A61K 8/64

(52) U.S. Cl. CPC A61K 8/975; A61K 8/64; A61K 8/97; A61K 8/55; A61Q 19/08; A61K 2800/70

(57) ABSTRACT

A topical composition and a method for treating skin to boost collagen production are provided. The composition comprises a collagen-boosting effective amount of a mixture of acetyl hexapeptide-3, Siegesbeckia orientalis extract, Laminaria digitata, whey protein, and magnesium ascorbyl phosphate.

### FIELD OF THE INVENTION

The present invention is related to the field of anti-aging of skin. More specifically, the invention concerns ingredients and compositions for treating skin to promote collagen production.

### BACKGROUND OF THE INVENTION

Collagen, a family of structurally related proteins, takes the form of elongated fibrils, and is the most abundant protein in the body. It is the main structural protein in connective tissues such as skin, and it is a principal component of the extracellular matrix of skin. The form of collagen in the skin forms loosely woven fibers. This fibrous protein possesses great tensile strength and serves to provide the support structure of the skin. Collagen, together with elastin, another main structural protein in the skin, gives the skin form and provides firmness and strength. Unlike most other proteins in the body, collagen can be found both inside and outside cells, and contributes to the external structure of cells.

As people age, however, collagen tends to degrade and collagen production decreases, leading to a loss of tone or tensile strength and elasticity, which results in sagging skin and the formation of wrinkles. Most of these age-dependent changes result from changes in fibroblasts which are responsible for collagen and elastin production. Fibroblasts produce collagen and elastin at a slower rate. The organization of these proteins changes, as the proteins become thicker, clumped and more loosely connected. The result is decreased elasticity and dryness or brittleness of the skin. Elevation of certain enzymes, collagenase and elastase, in aging skin, are largely responsible for the changes.

For those persons who already show signs of aging, either intrinsic (natural) aging, or extrinsic aging due to genetic factors, cumulative sun damage (photoaging), or chemical exposure, various treatments are available, including Vitamin A-derived substances, such as topical retinoic acid or Retin-A; natural products, such as alpha hydroxy acids and anti-oxidants; exposure to light treatments, such as near infra-red (NIR) light; and more aggressive treatments such as dermabrasion, injections with Botulinum toxin, chemical peels, laser resurfacing, face lifts, and so forth. Nevertheless, many of these treatments have been associated with significant side effects. There remains a need for cosmetic products which are both safe and which demonstrate efficacy in preventing, slowing and/or reversing the visible signs of aging on the skin.

### SUMMARY OF THE INVENTION

The present invention is directed to a composition for topical application to skin comprising a collagen-boosting effective amount of a mixture of acetyl hexapeptide-3 (Argireline), Siegesbeckia orientalis extract, Laminaria digitata (Mitostime), whey protein (NXP-75), and magnesium ascorbyl phosphate (MAP), in a cosmetically acceptable vehicle.

The present invention also concerns a method for treating skin to boost collagen production, the method comprising applying to skin in need thereof a composition comprising a collagen-boosting effective amount of a mixture of acetyl hexapeptide-3, Siegesbeckia orientalis extract, Laminaria digitata, whey protein, and magnesium ascorbyl phosphate (MAP), in a cosmetically acceptable vehicle and retaining the composition in contact with the skin for a time sufficient to boost collagen production in the skin.

### BRIEF DESCRIPTION OF THE DRAWINGS

FIG. 1 is a graph depicting the collagen-boosting efficacy of the composition of the present invention including acetyl hexapeptide-3, Siegesbeckia orientalis extract, Laminaria digitata, whey protein, and magnesium ascorbyl phosphate.

FIG. 2 is a graph comparing the collagen-boosting efficacies of a composition of the present invention including acetyl hexapeptide-3, Siegesbeckia orientalis extract, Laminaria digitata, whey protein, and magnesium ascorbyl phosphate, and the same composition omitting the whey protein.

FIG. 3 is a graph depicting the collagen-boosting efficacy of Argireline.

FIG. 4 is a graph depicting the collagen-boosting efficacy of whey protein (NXP-75).

### DETAILED DESCRIPTION OF THE PREFERRED EMBODIMENTS OF THE INVENTION

Skin aging is influenced by many factors, including, but not limited to, ultraviolet radiation, environmental pollution, alcohol and tobacco abuse, heredity, lifestyle, body weight, repetitive facial movements, gravity, and sleeping positions. Within the skin, aging is associated with loss of fibrous tissue (degradation of extracellular matrix), decrease in rate of cellular renewal, loss of cellular hydration due to impaired skin barrier function, and loss of subcutaneous fat, all of which result in thinner skin and a loss of its youthful qualities.

The loss of collagen in the skin resulting from the breakdown of collagen by collagenase and the decrease in collagen production by fibroblasts, has been a target for the investigation of how the signs of aging may be preventing, slowed or reversed. To that end, the inventors have discovered a system including a unique combination of ingredients which synergistically demonstrate enhanced collagen production in fibroblasts.

The inventors' system for boosting collagen production in skin includes a unique combination of natural and synthetic ingredients consisting of acetyl hexapeptide-3 (Argireline), Siegesbeckia orientalis extract, magnesium ascorbyl phosphate (MAP), whey protein (NXP-75) and Laminaria digitata Mitostime.

Argireline (acetyl hexapeptide-3) is a synthetic molecule which is said to mimic one of the proteins involved in muscle contraction and can destabilize it so that it will not function. Use of Argireline could eliminate one of the causes of wrinkling. Argireline has not been observed to boost collagen production or to repair the skin.

Siegesbeckia orientalis extract is obtained from Siegesbeckia orientalis L., an herb which has been observed to sooth inflammation, stimulate wound healing, and provide protection from UV induced erythema when topically applied to skin. The extract is said to act by building the collagen matrix, restoring elasticity to damaged skin by rearranging collagen fibers to restructure tissue, and inhibiting collagenase. The extract also is said to have potential as a treatment for scars and stretch marks.

Fibroblasts are the cells that help to maintain the structural integrity of connective tissues, including skin; however, as they age, they produce less and less collagen, which contributes to the visible signs of aging, including sagging skin and wrinkles. Mitostime® is a concentrated brown algae extract of Laminaria digitata that has been observed to stimulate the production of collagen by aging fibroblasts—even restoring fibroblast collagen production to youthful levels, protect mitochondrial DNA, boost the skin's use of oxygen and the release of more carbon dioxide, and reduce wrinkle depth.

MAP, a synthetic version of Vitamin C (L-ascorbic acid), is said to be a strong anti-oxidant and anti-aging ingredient. MAP is said to function similarly to L-ascorbic acid, a co-factor in the production of collagen, to help repair skin, and reduce the appearance of photo-damage, such as pigmentation marks (by inhibiting excessive melanin production), uneven texture and uneven tone.

Whey protein extract (such as NXP-75, obtained from cow's milk) is said to help the body heal and repair injured tissue, including preventing and treating osteoporosis. Whey protein contains proline, a glucogenic amino acid that is an essential component of collagen. Proline is said to speed up healing of damaged cartilage, help muscle growth and strengthen joints and tendons. Whey protein contains high concentrations of branched chain amino acids (BCAAs) including leucine, isoleucine, valine, arginine and glycine which are said to help promote healing of connective tissue, bones, skin and muscle.

Although each of the ingredients individually had been observed by the inventors to demonstrate some degree of collagen synthesis-promoting activity or an efficacy in repairing injured tissue, the inventors discovered that the ingredients in their system work synergistically to provide a greatly enhanced level of collagen-synthesis production which is surprising and unexpected based on their individual performances.

Compositions for topical application to skin contain a mixture of the following water-soluble ingredients: acetyl hexapeptide-3 (Argireline), preferably, in amounts in the range of from about 0.000005% to about 0.01% (w/v); Siegesbeckia orientalis extract, preferably in amounts in the range of from about 0.0005% to about 2.5% (w/v); Laminaria digitata (Mitostime), preferably, in amounts in the range of from about 0.0001% to about 5%; (w/v) magnesium ascorbyl phosphate (MAP), preferably, in amounts in the range of from about 0.00001% to about 5% (w/v); and whey protein (NXP-75), preferably, in amounts in the range of from about 0.0001% to about 5% (w/v). More preferably, the compositions contain acetyl hexapeptide-3 (Argireline) in amounts in the range of from about 0.00005% to about 0.0085% (w/v); Siegesbeckia orientalis extract in amounts in the range of from about 0.005% to about 1% (w/v); Laminaria digitata (Mitostime) in amounts in the range of from about 0.001% to about 3% (w/v); magnesium ascorbyl phosphate (MAP) in amounts in the range of from about 0.0001% to about 2% (w/v); and whey protein (NXP-75) in amounts in the range of from about 0.001% to about 5% (w/v).

Compositions according to the invention may include one or more additional ingredients, including, but not limited to, oils, pigments, powders, surfactants, structuring agents, sunscreens, preservatives, vitamins, anti-oxidants, botanicals, moisturizers, humectants, and fragrances.

Products incorporating the systems of the invention have anti-aging, firming, contouring and/or lifting efficacies for the skin around the eyes, facial skin, the skin of the hands and the body. Product forms may include, but are not limited to, an emulsion, a solution or a suspension, and may further take the form of, for example, a mask, a lotion, a day or night repair serum, a mousse, a day and/or night cream, a toner, a makeup foundation primer, a makeup foundation, and the like.

The present invention is also directed to a method for treating skin to boost collagen production. The method comprises applying to the skin in need of collagen boosting a composition comprising a collagen-boosting effective amount of a mixture of acetyl hexapeptide-3, Siegesbeckia orientalis extract, Laminaria digitata, whey protein, and magnesium ascorbyl phosphate. The composition is retained in contact with the skin for a time sufficient to boost collagen production in the skin.

Products incorporating a composition according to the present invention may be applied to any skin areas which have developed lines and/or wrinkles, or any skin areas which are susceptible to the adverse effects of the environment, daily stress, sun exposure, or premature aging, and which may be expected to develop lines and/or wrinkles. The products are particularly suited to address those skin areas which are most resistant to skin remodeling, such as the crow's feet in the skin of the periorbital regions around the eyes.

A composition of the present invention may be applied to the skin on an as-needed basis or according to a pre-set schedule. The composition may be applied directly to clean skin, before application of any other treatment product, or foundation makeup, or they may be applied over the other treatment product or foundation makeup. The amount of the composition applied to the skin with each application can vary widely depending on the specific need of the user. For example, if the user has prominent wrinkles, the user may choose to apply the compositions more frequently than if the user's skin exhibits finer lines. The composition may be applied for a period of days to months or even years, and at a frequency ranging from about once or twice a day to once a week. In another example, the composition may be applied one or twice a day, morning and/or evening, for a period of six months or more.

The invention will be further described by the following non-limiting examples which are provided for the purposes of illustration only.

### EXAMPLES

### Example 1

### Collagen-Boosting Assay

To observe the effect of a unique combination of actives on collagen synthesis, adult normal human dermal fibroblasts (NHDFs) were treated with a mixture of Argireline, Siegesbeckia orientalis extract, MAP, NXP-75 and Mitostime (Laminaria digitata extract) in growth medium ((Dulbecco's Modified Eagle Medium/10% Fetal Bovine Serum/1% Penicillin (5000 units)/1% Streptomycin (5000 mg/ml)) at the concentrations shown below in Table 1. The undiluted mixture contains 0.01 mg/ml Argireline, 2.5 mg/ml Siegesbeckia, and 1 mg/ml of each of MAP, NXP-75 and Mitostime in growth medium.

TABLE 1
(%)

| Collagen-boosting mixture | undiluted | | | | | | | |
|---|---|---|---|---|---|---|---|---|
| ingredients | (UD) | 1:2 | 1:4 | 1:8 | 1:16 | 1:32 | 1:64 | 1:128 |
| Argireline | 0.001 | 0.0005 | 0.00025 | 0.000125 | 0.0000625 | 0.00003125 | 0.000015625 | 0.000007812 |
| Siegesbeckia orientalis | 0.25 | 0.125 | 0.0625 | 0.03125 | 0.015625 | 0.0078125 | 0.00390625 | 0.001953125 |
| extract | | | | | | | | |
| Magnesium Ascorbyl | 0.1 | 0.05 | 0.025 | 0.0125 | 0.00625 | 0.003125 | 0.0015625 | 0.00078125 |
| Phosphate | | | | | | | | |
| NXP-75 | 0.1 | 0.05 | 0.025 | 0.0125 | 0.00625 | 0.003125 | 0.0015625 | 0.00078125 |
| Mitostime Powder | 0.1 | 0.05 | 0.025 | 0.0125 | 0.00625 | 0.003125 | 0.0015625 | 0.00078125 |
| Growth Medium (ml) | 99.449 | 99.7245 | 99.86225 | 99.931125 | 99.9655625 | 99.89278125 | 99.99139603 | 99.99569531 |

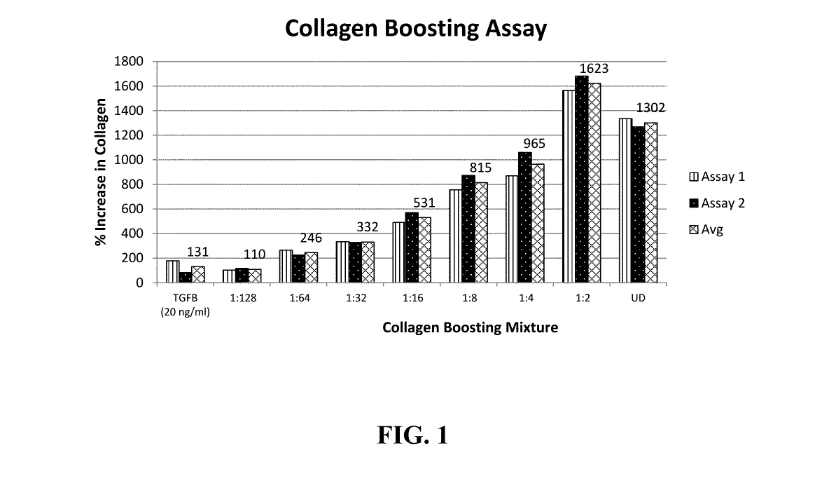

The NHDFs were seeded and grown to confluence in a 96 well plate. NHDFs were treated with the various concentrations of the mixture or with media and incubated for 3 days at 37° C./5% CO₂ with. The supernatants were then harvested and stored at −80° C. in siliconized tubes prior to being analyzed for collagen release using a Procollagen Type 1C-peptide enzyme immunoassay (PIP EIA or PIP ELISA). Supernatants were diluted 1:200 for the assay. TGFβ, at 20 ng/ml was run as a positive control. The assay was performed in accordance with the manufacturer's standard protocol (Takara Minis Bio) and results calculated from the standard curves. The amount of PIP (pro-collagen ng/ml medium) is quantitated by measuring the absorbance using an EIA plate reader. Accurate sample concentrations of PIP can be determined by comparing the specific absorbances to a standard curve. Cell viability also was assessed using a standard 3-(4,5-dimethylthiazol-2-yl)-2,5-diphenyltetrazoliumbromide (MTT) assay. The assay involves the conversion of the water soluble MTT, a yellow tetrazole, to an insoluble purple formazan in living cells. The formazan is then solubilized and the concentration determined by measuring the absorbance at a wavelength of from about 500-600 nm using a spectrophotometer. The Collagen-boosting assay was repeated. Results of Assay 1 and Assay 2 are shown in FIG. 1.

The following calculation was employed to ascertain the percent increase in collagen production resulting from treatment of NHDFs with the mixture or with the positive control (TGFβ), as compared with that amount of collagen produced using media:

{[(% collagen increase of test sample)−(% collagen increase for media alone)]/(% collagen increase for media alone)}×100.

As indicated in the graph in FIG. 1, the collagen-boosting mixture increased collagen synthesis in a dose dependent manner. The percent increase in collagen synthesis (over the percent increase for media alone), demonstrates that the collagen-boosting mixture is effective over a wide range of concentrations and, at most concentrations, surprisingly exceeds the amount of collagen synthesized by the NHDFs incubated with the positive control, TGFβ. Results observed are statistically significant as shown in Tables 2 and 3, below.

TABLE 2
p values: 2 tailed T test:
mixture and TGFβ v. media

| | Assay 1 | Assay 2 |
|---|---|---|
| TGFβ | 0.000312 | 3.3E−05 |
| 1:128 | 0.00057 | 0.002688 |
| 1:64 | 6.2E−06 | 1.97E−05 |
| 1:32 | 0.001736 | 0.000283 |
| 1:16 | 0.001257 | 0.00533 |
| 1:8 | 1.39E−05 | 0.014282 |
| 1:4 | 0.000459 | 0.003071 |
| 1:2 | 0.002963 | 0.007766 |
| UD | 0.002439 | 0.002442 |

TABLE 3
p values: 2 tailed T test:
mixture v. TGFβ

| | Assay 1 | Assay 2 |
|---|---|---|
| 1:128 | 0.079297 | 5.54E−05 |
| 1:64 | 0.000146 | 0.000969 |
| 1:32 | 0.000772 | 0.033241 |
| 1:16 | 0.007593 | 0.008727 |
| 1:8 | 0.016332 | 0.000147 |
| 1:4 | 0.007214 | 0.001411 |
| 1:2 | 0.00883 | 0.004469 |
| UD | 0.002937 | 0.00412 |

### Example 2

### Collagen-Boosting Assay

To further demonstrate the synergistic collagen-boosting activity of the mixture of the present invention on NHDFs, the collagen-boosting assay was performed using the mixture of the invention (as used in Example 1) and the same collagen-boosting mixture which omitted NXP-75. TGFβ was run as a positive control.

Adult NHDF cells were seeded and grown to confluence in a 96 well plate prior to being treated. Both mixtures were tested at 8 concentrations (undiluted, and at 1:2, 1:4, 1:8, 1:16, 1:32, 1:64 and 1:128). The plate was incubated for 3 days at 37° C./5% CO₂ before the supernatants were harvested and stored at −80° C. in siliconized tubes until the pro-collagen (PIP) ELISA was performed, as described in Example 1. For this PIP ELISA, the supernatants were diluted 1:400. The assay was performed in accordance with the manufacturer's standard protocol.

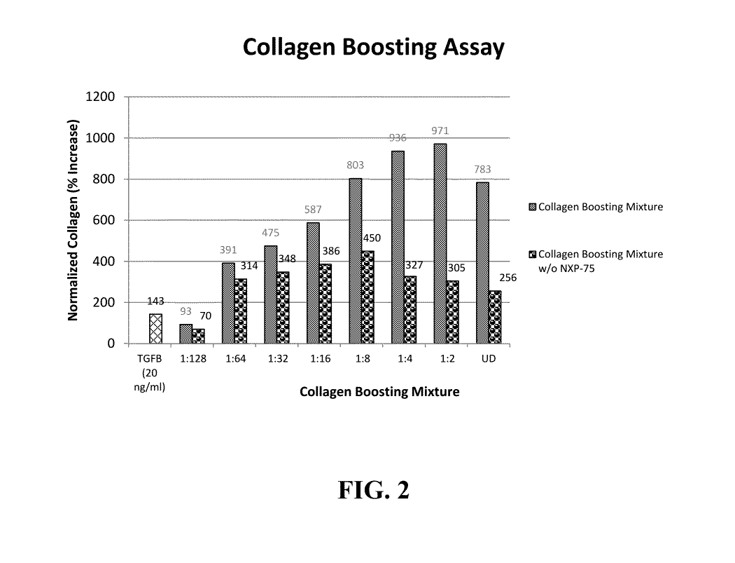

Results are shown in FIG. 2. As indicated in the graph, over a wide range of concentrations, the mixture according to the present invention outperformed the mixture which lacked the NXP-75. Results observed are statistically significant as shown in Tables 4 and 5, below.

TABLE 4
p values: 2 tailed T test: mixture
with and w/o NXP-75; TGFβ v. media

| | w/ NXP- | w/o |
|---|---|---|
| | 75 | NXP-75 |
| TGFβ | | 0.005723 |
| 1:128 | 0.002375 | 0.001317 |
| 1:64 | 0.000499 | 5.8E−06 |
| 1:32 | 1.99E−09 | 0.000239 |
| 1:16 | 0.004084 | 0.002745 |
| 1:8 | 0.000626 | 0.000604 |
| 1:4 | 0.000378 | 0.001876 |
| 1:2 | 0.002916 | 0.000585 |
| UD | 7.72E−05 | 0.004516 |

TABLE 5
p values: 2 tailed T test: mixture
with and w/o NXP-75 v. TGFβ

| | | mixture |
|---|---|---|
| | | w/o |
| | mixture | NXP-75 |
| 1:128 | 0.029984 | 0.021513 |
| 1:64 | 0.000452 | 0.002211 |
| 1:32 | 0.001547 | 0.000271 |
| 1:16 | 0.004124 | 0.001689 |
| 1:8 | 6.82E−05 | 3.43E−05 |
| 1:4 | 1.94E−05 | 0.000631 |
| 1:2 | 0.002774 | 0.000155 |
| UD | 1.94E−05 | 0.008453 |

### Example 3

### Collagen-Boosting Assay

In this assay, Argireline was tested for its efficacy in boosting collagen synthesis in NHDFs. TGFβ was run as a positive control.

Adult NHDFs were seeded and grown to confluence in a 96 well plate prior to treatment with Argireline at concentrations of 0.001, 0.01, 0.1 and 1% (v/v) in media (negative control) or with 10 ng/ml TGFβ (positive control) in media.

The plate was incubated for 3 days at 37° C./5% CO₂. Supernatants were harvested and stored at −80° C. in siliconized tubes until the PIP ELISA was performed, as described in Example 1. For this assay, supernatants were diluted 1:40.

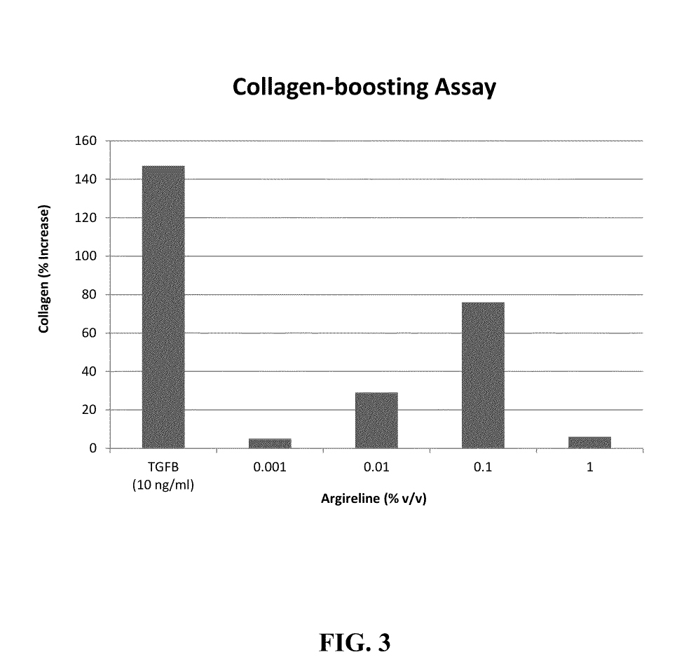

The results, shown in FIG. 3, demonstrate that Argireline induces collagen synthesis in NHDFs. At 0.1% (v/v), Argireline increased collagen synthesis by a 76% compared with media. At 0.01%, Argireline increased collagen synthesis by a 29% compared with media. At 10 ng/ml, the positive control, TGFβ, was observed to increase collagen levels by 147%.

### Example 4

### Collagen-Boosting Assay

Mitostime was tested for its collagen-boosting efficacy in NHDFs. TGFβ, at 10 ng/ml, was run as a positive control. Adult NHDFs were seeded and grown to confluence in a 96 well plate prior to treatment with mitostime 0.075%, 0.15% and 0.3% (w/v). The plate was incubated for 3 days at 37° C./5% CO₂. Supernatants were harvested and stored at −80° C. in siliconized tubes until the PIP ELISA was performed as outlined in Example 1. For this assay, supernatants were diluted 1:200. At 0.3%, 0.15% and 0.075%, Mitostime increased collagen synthesis by 158% (p=6.36×10⁻⁷), 159% (p=0.0005) and 122% (p=3.682×10⁻⁵), respectively, compared with media (negative control). TGFβ increased collagen synthesis by 27% (p=0.0001).

Mitostime also was tested in combination with Argireline for efficacy in boosting collagen synthesis in NHDFs. TGFβ, at 10 ng/ml, was run as a positive control. Adult NHDFs were seeded and grown to confluence in a 96 well plate prior to treatment with Mitostime at 0.3%/Argireline peptide at 0.00005%; Mitostime at 0.15%/Argireline peptide at 0.000025%; and Mitostime at 0.075%/Argireline peptide at 0.0000125%. The plate was incubated for 3 days at 37° C./5% CO₂. Supernatants were harvested and stored at −80° C. in siliconized tubes until the PIP ELISA was performed as outlined in Example 1. For this assay, supernatants were diluted 1:200. The combination of Mitostime and Argireline was found to induce greater collagen synthesis in NHDFs compared to Mitostime or Argireline when used without the other. Collagen synthesis was increased by 200% (p=2.4×10⁻⁵), 229% (p=2.56×10⁻⁵) and 210% (p=1.51×10⁻⁵) for the following combinations: Mitostime at 0.3%/Argireline peptide at 0.00005%; Mitostime at 0.15%/Argireline peptide at 0.000025%; and Mitostime at 0.075%/Argireline peptide at 0.0000125%, respectively. TGFβ increased collagen synthesis by 23%.

### Example 5

### Collagen-Boosting Assay

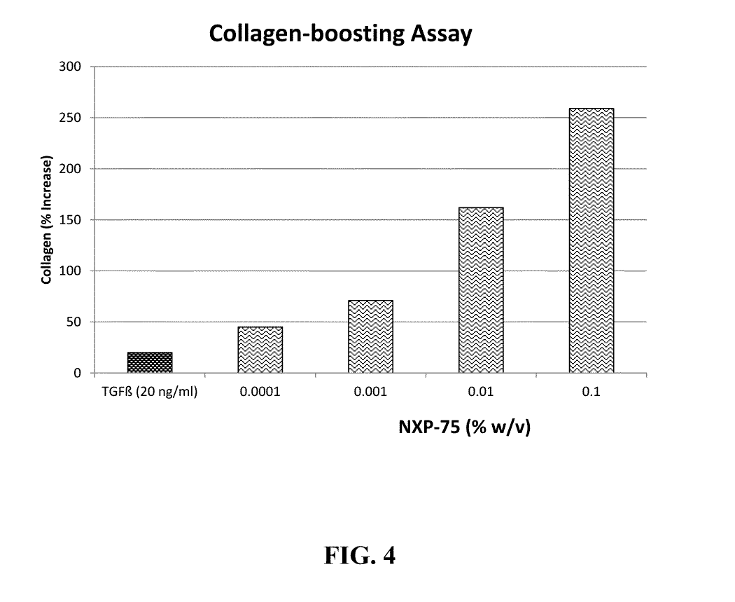

In this assay, NXP-75 was tested for its efficacy in boosting collagen synthesis in NHDFs. TGFβ (10 ng/ml) was run as a positive control. Adult NHDF cells were seeded and grown to confluence in a 96 well flat bottom plate prior to treatment. NXP-75 powder was prepared at the following dilutions: 0.0001, 0.001, 0.01 and 0.1% (w/v) in media (negative control). The plate was incubated for 3 days at 37° C./5% CO₂. Supernatants were harvested and stored at −80° C. in siliconized tubes until the PIP ELISA was performed. For this assay, supernatants were diluted 1:200. As shown in FIG. 4, at 0.0001%, NXP-75 boosts collagen synthesis in NHDF cells by 45% (p=0.0002) compared with media; at 0.001%, NXP-75 boosts collagen synthesis by 71% (p=6.58×10⁻⁵); at 0.01%, NXP-75 boosts collagen synthesis by 162% (p=0.0007); and at 0.1%, NXP-75 boosts collagen synthesis by 259% (p=4.05×10⁻⁶). TGFβ enhanced collagen synthesis by 20% compared with media.

While some illustrative embodiments of the invention have been described hereinabove, such illustrative embodiments should not be interpreted in any manner to limit the broad scope of the present invention. Various modifications and equivalents of the described embodiments and components thereof will be apparent to those of ordinary skill in the art. Some modifications and equivalents will be readily recognized by one ordinarily skilled in the art, while others may require no more than routine experimentation. It is therefore understood that such modifications and equivalents are within the spirit and scope of the present invention.

What we claim is:

1. A composition for topical application to skin comprising a collagen-boosting effective amount of a mixture of acetyl hexapeptide-3, Siegesbeckia orientalis extract, Laminaria digitata, whey protein, and magnesium ascorbyl phosphate, in a cosmetically acceptable vehicle.

2. The composition of claim 1, comprising acetyl hexapeptide-3, in amounts in the range of from about 5×10⁻⁶% to about 0.01% (w/v); Siegesbeckia extract, in amounts in the range of from about 5×10⁻⁴% to about 2.5% (w/v); Mitostime, in amounts in the range of from about 0.0001% to about 5%; (w/v) magnesium ascorbyl phosphate, in amounts in the range of from about 0.00001% to about 5% (w/v); and whey protein in amounts in the range of from about 0.0001% to about 5% (w/v).

3. The composition of claim 2, comprising acetyl hexapeptide-3 in amounts in the range of from about 0.000005% to about 0.0085% (w/v); Siegesbeckia extract in amounts in the range of from about 0.005% to about 1% (w/v); Laminaria digitata in amounts in the range of from about 0.001% to about 3% (w/v); magnesium ascorbyl phosphate in amounts in the range of from about 0.0001% to about 2% (w/v); and whey protein in amounts in the range of from about 0.001% to about 5% (w/v).

4. The composition of claim 1, comprising at least one ingredient selected from oils, pigments, powders, surfactants, structuring agents, sunscreens, preservatives, vitamins, anti-oxidants, botanicals, moisturizers, humectants, and fragrances.

5. The composition of claim 1, in a form selected from a mask, a lotion, a day or night repair serum, a mousse, a day and/or night cream, a toner, a makeup foundation primer, and a makeup foundation.

6. A method for treating skin to boost collagen production, the method comprising applying to skin in need thereof a composition comprising a collagen-boosting effective amount of a mixture of acetyl hexapeptide-3, Siegesbeckia orientalis extract, Laminaria digitata, whey protein, and magnesium ascorbyl phosphate, in a cosmetically acceptable vehicle, and retaining the composition in contact with the skin for a time sufficient to boost collagen production in the skin.

7. The method of claim 6, wherein the composition comprises acetyl hexapeptide-3 in amounts in the range of from about 0.00005% to about 0.0085% (w/v); Siegesbeckia extract in amounts in the range of from about 0.005 to about 1% (w/v); Laminaria digitata in amounts in the range of from about 0.001% to about 3% (w/v); magnesium ascorbyl phosphate in amounts in the range of from about 0.0001% to about 2% (w/v); and whey protein in amounts in the range of from about 0.001% to about 5% (w/v).

8. The method of claim 7, wherein the composition comprises acetyl hexapeptide-3 in amounts in the range of from about 0.00005% to about 0.0085% (w/v); Siegesbeckia extract in amounts in the range of from about 0.005% to about 1% (w/v); Laminaria digitata in amounts in the range of from about 0.001% to about 3% (w/v); magnesium ascorbyl phosphate in amounts in the range of from about 0.0001% to about 2% (w/v); and whey protein in amounts in the range of from about 0.001% to about 5% (w/v).

9. The method of claim 6, wherein the composition comprises at least one ingredient selected from oils, pigments, powders, surfactants, structuring agents, sunscreens, preservatives, vitamins, anti-oxidants, botanicals, moisturizers, humectants, and fragrances.

10. The method of claim 6, wherein the composition is in a form selected from a mask, a lotion, a day or night repair serum, a mousse, a day and/or night cream, a toner, a makeup foundation primer, and a makeup foundation.
